(19) United States
(12) Patent Application Publication
(10) Pub. No.: US 2026/0102343 A1
(43) Pub. Date: Apr. 16, 2026

(54) INJECTABLE EMULSION FORMULATIONS OF CLEVIDIPINE

(71) Applicant: GOOD HEALTH, LLC, Princeton, NJ (US)

(72) Inventor: Basavaraj Siddalingappa, Gujarat (IN)

(73) Assignee: GOOD HEALTH, LLC, Princeton, NJ (US)

(21) Appl. No.: 19/357,973

(22) Filed: Oct. 14, 2025

Related U.S. Application Data
(60) Provisional application No. 63/707,432, filed on Oct. 15, 2024.

(51) Int. Cl. A61K 9/107; A61K 9/00; A61K 31/4418; A61K 47/10; A61K 47/12; A61K 47/14; A61K 47/20; A61K 47/22; A61K 47/24; A61K 47/44

(52) U.S. Cl. CPC A61K 9/1075; A61K 9/0019; A61K 31/4418; A61K 47/10; A61K 47/12; A61K 47/14; A61K 47/20; A61K 47/22; A61K 47/24; A61K 47/44

(57) ABSTRACT

Injectable Clevidipine emulsion formulations having enhanced stability and clarity for extended periods of time are disclosed. Methods of making the compositions as well as compositions made by the method and methods of treatment using the compositions are also disclosed.

### CROSS-REFERENCE TO RELATED APPLICATIONS

This application claims the benefit of priority from U.S. Provisional Application No. 63/707,432 filed Oct. 15, 2024, the contents of which are incorporated herein by reference.

### FIELD OF THE INVENTION

This invention relates stable, physiologically compatible emulsion formulations of Clevidipine intended for intravenous injection.

### BACKGROUND OF THE INVENTION

Clevidipine is an anti-hypertensive drug used to treat patients with elevated blood pressure. Cleviprex is indicated for the reduction of blood pressure when oral therapy is not feasible or not desirable.

Clevidipine is a dihydropyridine calcium channel blocker that reduces blood pressure in a subject to which it is administered. It is characterized as a short-acting, highly selective drug that is typically used in a hospital setting due to its rapid metabolism, exhibiting an initial phase half-life of about one minute and a terminal half-life of approximately 15 minutes.

Clevidipine is further characterized by having low solubility in water and moderate to high solubility in lipids. Due to its insoluble nature, Clevidipine needs to be formulated as emulsion for intravenous injection. Moreover, due to rapid metabolism, intravenous injection needs to be continuous infusion to maintain desired concentration in the blood.

U.S. Pat. No. 8,658,676 discloses an injectable pharmaceutical emulsion formulation of Clevidipine containing EDTA as an antimicrobial agent. In addition, the formulation consists of lipid, emulsifier, tonicity modifier and water, wherein pH of the formulation is between 6-8.0. The formulations claim to have resistance to formation of impurities in addition to resistance to microbial growth.

U.S. Pat. No. 10,010,537 discloses emulsion formulations of Clevidpine for injection with EDTA as an antimicrobial agent, soyabean oil as a lipid component, purified egg yolk solids as an emulsifier, glycerol as a tonicity adjusting agent and oleic acid as a co-emulsifier and stabilizer.

U.S. Pat. No. 11,103,490 also discloses injectable emulsions of clevidipine using soyabean oil, egg yolk solids, glycerol, oleic acid and EDTA as an antimicrobial preservative. However, it additionally mentions use of antioxidants in the formulations such as sodium ascorbate, sodium citrate, cysteine, sodium sulphite, ascorbyl palmitate, propyl gallate but patent does not disclose any example using these antioxidants and their beneficial effect on the stability of Clevidipine in the emulsion formulation.

What is not disclosed in the above patents is that Clevidipine emulsions that are stable at ambient and higher temperature for extended periods, have pH closer to blood and can be autoclaved in commonly used autoclaves which do not have an option of rotation/mixing during the autoclaving process. The present invention addresses these shortcomings.

### SUMMARY OF THE INVENTION

This invention relates to Clevidipine injectable emulsions suitable for intravenous administration including those suitable as large volume parenterals in glass containers and which are autoclaved. The clevidipine emulsion formulations described herein are preferably free of EDTA and its salts and the inventive emulsion formulations exhibit enhanced stability at ambient conditions.

This invention also describes a method of treatment of patients in need of treatment with the clevidipine compositions of the invention, such as patients with elevated blood pressure. Such methods include administering an effective amount of the clevidipine-containing compositions as described herein to a patient in need of such drug, preferably by administering the composition as intravenous IV infusion without further dilution.

The invention also relates to methods of making Clevidipine injectable emulsion compositions described herein, the clevidipine emulsion compositions made by the methods, and methods of packing compositions of this invention into suitable vials and terminally sterilizing the vials.

It has been surprisingly found that when sufficient amounts of cysteine, i.e. L-cysteine, designated herein as stabilizing amounts, are added after final milling of the emulsion or one or more cycles prior to the final milling to form the nanoemulsion, such as with a microfluidizer or similar device, the resulting clevidipine emulsion formulations have enhanced stability. The stability is evidenced by not only improved stability of the clevidipine but also improved physical stability of the emulsions, as well as globule size reduction, maintaining pH during autoclaving. In most aspects of the invention, stabilizing amounts of L-cysteine are from about 0.1 to about 0.5 mg/mL, or from about 0.001 to 0.05% W/W cysteine, based upon the total weight of the emulsion. amount Alternatively, the cysteine can be added to the coarse or first emulsion formed when the oil and water phases are combined, or before some or all of the further milling cycles are caried out to complete the formulation.

For purposes of the present invention, it will be understood that the term “coarse emulsions” is used in the manner understood by those of ordinary skill in the art. As such, “coarse emulsions” are the are preliminary mixtures of oil and water phases with relatively large droplet sizes (typically >200 nm) created using simple mixing, such as with a high-shear mixer, before being subjected to high-energy techniques like microfluidization or high-pressure homogenization. Coarse emulsions shall therefore be further understood as including emulsions in which the droplet sizes exceed the upper limit of the desired size range which is achieved after a sufficient number, e.g. about 5, of milling or microfluidization cycles.

### DETAILED DESCRIPTION OF THE INVENTION

The invention relates to stable, liquid pharmaceutical compositions comprising Clevidipine, at least one lipid excipient, an oil, a surfactant, a co-surfactant, organic salts, solvent, antimicrobial preservative, antioxidants and tonicity agent. Within this broad aspect of the invention, some inventive compositions include Clevidipine injectable emulsion formulations which include Clevidipine, an oil, a phosphatidylcholine emulsifier, a coemulsifier, a cosolvent, preservative, antioxidant and water, a pH modifier and tonicity adjuster. The Clevidipine-containing emulsions are oil in water emulsions. The inventive emulsion formulations are suitable for IV (intravenous) administration to patients in need of treatment or treatment of elevated blood pressure that needs to be brought in control as quickly as possible.

Preferably, the Clevidipine is in its ester form or its pharmaceutically acceptable salt thereof. The term “Clevidipine” includes Clevidipine Butyrate or a pharmaceutically acceptable salt, other esters, or prodrug thereof. The Clevidipine may alternatively be in the form of a complex. The Clevidipine may be present in the compositions of the invention in any amount, such as an amount ranging from about 0.01% W/W to about 0.1% W/W.

The emulsifier present in compositions of this invention are lecithins and are more preferably predominantly phosphatidylcholines which may be derived from natural, semi-synthetic or synthetic source but are in most aspects of the invention obtained from natural sources. Suitable phosphatidylcholines used in the inventive compositions are either from soya or egg lecithin. In addition, the phosphatidylcholine (PC) included in the compositions of this invention are pharmaceutically acceptable PC's and purified forms of lecithin, with phosphatidylcholine content >80%, preferably >85% W/W, more preferably >90% W/W, most preferably >92% W/W.

The amount of emulsifier, i.e., PC, present in the formulation is preferably <about 2% W/W. In some aspects of the invention, the amount of the phosphatidylcholine emulsifier is from about 1.0 to about 1.6% W/W of the formulation. In further aspects, the amount of PC in the emulsion is > (i.e. greater than) about 0.8% W/W or >about 1% W/W, or >about 1.1% W/W, or about 1.2% W/W, but still <1.6% W/W.

The injectable Clevidipine emulsion formulations of the invention also include an oil which is preferably a vegetable oil such as soya bean oil in amount of from about 15 to about 25% W/W of the formulation. In alternative aspects, the amount of vegetable oil in the composition is from about 18 to about 20% W/W.

The compositions of this invention may optionally contain a surfactant belonging to synthetic or semi-synthetic class such as an ester of a fatty acids or ester of vitamin E or a ester of phospholipid such as either derivative of vitamin E or phospholipid like Vitamin TPGS or Pegylated phospholipids such as 1,2-distearoyl-sn-glycero-3-phosphoethanolamine-N-[amino (polyethylene glycol)-2000] Na, DSPE-PEG2000, Na or Vitamin E TPGS or Polysorbate 80.

The emulsion compositions of the invention will also contain a cosolvent, which is an organic solvent such as short chain alcohol, like glycerol or glycerin (e). For purposes of the invention, glycerol, glycerin and glycerine are used interchangeably. Other cosolvents useful in the formulations of the present invention include polyethylene glycol, propylene glycol or mixtures thereof, or optionally combined with glycerol. Preferably, the organic cosolvent is glycerol. The amount of the cosolvent in the formulation is from about 1 to about 5% W/W of the formulation in most embodiments. Alternatively, the amount of organic solvent in the composition can be from about 1 to about 4% W/W, or from about 2% W/W to about 3.0% W/W.

The amount of water in the injectable Clevidipine emulsion formulations described herein can be from about 65 to about 80% W/W of the formulation, with amounts of about 70-76% W/W in some embodiments.

The co-emulsifier included in the injectable Clevidipine emulsion formulations can be a fatty acid or salt of a fatty acid such as the sodium salts thereof, i.e., Oleic acid or sodium oleate. In many aspects of the invention, the amount of the co-emulsifier is from about 0.01 to about 0.04% W/W of the formulation. In alternative aspects, the amount of coemulsifier is from about 0.02 to about 0.04% W/W, or from about 0.02 to about 0.03% W/W.

The compositions of this embodiment have pH value of at least 5. In some embodiments the pH is in the range of between 5-8, preferably between 6 to 7.5.

The antioxidants in the Clevidipine injectable emulsion which are included in the oil phase may include ascorbyl palmitate, tocopherol, BHA, BHT, or vitamins or mixtures thereof. Preferably, the oil phase antioxidant is tocopherol.

The concentration of oil phase antioxidants in the formulation may be from 0.001 to 0.05% W/W, preferably from 0.01 to 0.05% W/W, or from 0.01 to 0.02% W/W.

The antimicrobial preservative in the composition may include but is not limited to methyl paraben, propyl paraben, benzalkonium chloride, benzyl alcohol, sodium benzoate or mixture thereof. Sodium benzoate and methyl paraben are preferred.

The concentration of preservative in the formulation may be from 0.001 to 0.05% W/W, preferably from 0.01 to 0.05% W/W.

Tonicity agents suitable for inclusion in the Clevidipine compositions of the invention include tonicity adjusting agents such as sodium chloride, dextrose, lactose, mannitol, sorbitol, and sucrose. In some preferred aspects, when the cosolvent is glycerol, the tonicity agent is sucrose.

The concentration of the tonicity agent is from about 1 to about 4% W/W, with amounts of from about 1 to about 2.5% W/W being preferred.

In view of the foregoing, some suitable formulations in accordance with the invention may include:
a) from about 0.01 to about 0.1% W/W Clevidipine Butyrate;
b) from about 15 to about 25% W/W soya bean oil;
c) from about 1 to about 1.6% W/W of egg or soya phosphatidylcholine emulsifier;
d) from about 1 to about 5% W/W of Glycerol;
e) from about 65 to about 80% W/W of water;
f) from about 0.01 to about 0.04% W/W sodium oleate or Oleic acid;
g) optionally from about 0.01 to about 0.05% W/W Tocopherol;
h) from about 0.001 to 0.05% W/W Cysteine and, optionally
i) from about 0.001 to 0.05% W/W preservative.

In accordance with a further aspect of the invention there are provided methods of preparing the Clevidipine emulsion compositions described herein. For example, one suitable method comprises:
a) providing an aqueous phase which includes water and optional ingredients such as tonicity agent, pH adjusting agents, buffers, emulsifier, cosolvent other pharmaceutically acceptable excipients useful in emulsion formation; in some embodiments, buffer or pH modifier can be included in the oil phase;
b) providing an oil phase which includes the oil, Clevidipine, oleic acid and in some embodiments, the oil soluble antioxidant, e.g. (alpha) tocopherol; and
c) combining the oil phase with the aqueous phase under conditions sufficient to form an emulsion, wherein the emulsion has an initial pH of at least 7, or 7-8.

In some preferred aspects of the invention, the anti-oxidant, e.g. L-cysteine, can be 1) added to the nano-emulsion after coarse milling, 2) added to the emulsion just before last one or two milling cycles and formation of the finished microemulsion or 3) added to the final emulsion for enhanced stabilization of the formulation. The amount of oil phase antioxidant can optionally be reduced or eliminated when this aspect of the emulsion formation process is carried out.

In the first two embodiments described in the preceding paragraph, the method described above includes breaking the emulsion formation step into at least two parts: a first part in which a coarse emulsion, as defined hereinabove, is formed and then a second part in which the coarse emulsion is combined with a stabilizing amount of L-cysteine, preferably from about 0.1 to about 0.5 mg/ml, and subjected to high shear mixing such as being milled through a microfluidizer or other suitable emulsion forming apparatus to form a microemulsion wherein the composition droplets preferably have a mean globule size (Z-average) of from about 100 or about 130 to about 180 nm. In the third embodiment, the cysteine It has been surprisingly found that compositions made using this process have superior stability when compared to those compositions which do not include the addition of L-cysteine during the second step of the emulsion formation process.

In accordance with some aspects of the invention, some oil in water clevidipine-containing emulsions will comprise from about 0.01 to about 0.1% W/W Clevidipine Butyrate;
a) from about 15 to about 25% W/W soya bean oil;
b) from about 1 to about 1.6% W/W of egg or soya phosphatidylcholine emulsifier;
c) from about 1 to about 5% W/W of Glycerol;
d) from about 65 to about 80% W/W of water;
e) from about 0.01 to about 0.04% W/W sodium oleate or Oleic acid;
f) optionally from about 0.01 to about 0.05% W/W Tocopherol
g) from about 0.001 to 0.05% W/W Cysteine and optionally,
h) from about 0.001 to 0.05% W/W preservative;
   wherein the oil in water emulsion is formed by combining a water phase with a clevidipine-containing oil phase to form a first or coarse emulsion and the cysteine is added either to the final emulsion or during a second emulsion forming step where the coarse emulsion or an intermediate emulsion in which the droplet size has undergone some reduction in droplet size undergoes further milling or microfluidization to form the final clevidipine emulsion having a droplet size range of from about 100 to about 180 nm. The intermediate emulsions where the cysteine can be added are typically those emulsions in which one or two additional milling cycles are needed before the final composition is obtained.

A “stable” composition of the invention means a pharmaceutical composition having sufficient stability at room temperature conditions to have utility as a pharmaceutical product. Preferably, a “stable” composition of the invention has sufficient stability to allow storage at room temperature conditions, preferably between about 15° C. and about 30° C., more preferably about 20° C. to about 25° C., most preferably about 25° C., and between about 55% to about 65% RH (e.g., about 60% RH), for a reasonable period of time, e.g., the shelf-life of the product which can be as short as one month but is typically three months or longer. A “stable” composition of the invention also includes specific ranges of impurities as described herein. Preferably, a “stable” composition is one which has minimal degradation of the at least one drug, e.g., it retains at least about 90% of un-degraded active, preferably at least about 95%, more preferably at least about 99%, after storage at about 2-8° C. for a 1-to-3-year period of time. Stable composition also implies that composition retains physical stability with respect to desired globule size, zeta potential, and particulate matter.

TABLE 1
Some Preferred Compositions for Clevidipine injectable emulsions

| | | Preferred | | Range |
|---|---|---|---|---|
| Ingredients | Function | Quantity mg/mL | | (% W/W) |
| Clevidipine | API | 0.5 | mg | 0.01-0.1 |
| Butyrate | | | | |
| Egg | Emulsifier | 12 | mg | 1-1.6 |
| Phosphatidylcholine | | | | |
| Soyabean Oil | Oil phase | 200 | mg | 15-25 |
| Glycerin | Cosolvent | 22.5 | mg | 1-5 |
| Oleic Acid | Coemulsifier | 0.3 | mg | 0.01-0.04 |
| Sodium | Preservative | — | | 0.001-0.05 |
| Benzoate | | | | |
| Methylparaben | Preservative | 0.08 | mg | 0.001-0.05 |
| L-Cysteine | pH modifier and | 0.1 | mg | 0.01-0.5 |
| | stabilizer when | | | |
| | added to either | | | |
| | final emulsion | | | |
| | or close to final | | | |
| | milling cycle. | | | |
| Alpha | Antioxidant | 0.2 | mg | |
| tocopherol | | | | |

| | | | |
|---|---|---|---|
| Milli | Water phase | Q.S. | — |
| Q Water | | to 1 mL | |

### EXAMPLES

### Materials:

Clevidipine Butyrate (>99% purity) was procured from MSN Laboratories Private Limited, India. Phosphatidyl Choline (Egg), Soya bean oil were all procured from Lipoid GMBH, Germany. Oleic acid was procured was procured from Croda, USA. All other chemicals and reagents were of high purity grade and were procured from reputable local vendors.

### Methods:

### HPLC Method:

In absence of any compendial method for the determination potency and Method is as described below.

### Materials and Method:

### Materials:

Sodium dihydrogen phosphate, AR Grade or equivalent Phosphoric acid, AR Grade or equivalent
Acetonitrile, HPLC grade or equivalent
Methanol, HPLC grade or equivalent

### Mobile Phase Preparation

### Buffer:

Weigh and transfer about 1.56 g of sodium dihydrogen phosphate in 1000 mL water. Sonicate for 5 minutes to dissolve the contents. Adjust pH 3.0 with phosphoric acid.

### Mobile Phase A:

Prepare a mobile phase by combining 300 mL of buffer with 700 mL of acetonitrile to form a homogenous mixture. Filter the resulting solution through a 0.45 μm nylon membrane filter and subsequently degas.

### Mobile Phase B:

Prepare a mobile phase by combining 340 mL of buffer with 660 mL of methanol to form a homogeneous mixture. Filter the resulting solution through a 0.45 μm nylon membrane filter and subsequently degas.

### Diluent:

Prepare a homogeneous solution by combining 100 mL of water with 200 mL of Ethanol. Mix thoroughly until a uniform composition is achieved.

Clevidipine Standard Solution (0.17 mg/mL of Clevidipine):

Accurately weigh and transfer about 1.7 mg of Clevidipine Standard into a 10-mL volumetric flask. Add 7 mL diluent and sonicate for 2 minutes with intermittent shaking to dissolve the contents. Makeup to mark with diluent and mix well.

Placebo Solution (0.17 mg/mL of Clevidipine)

Transfer 1.0 mL of Clevidipine Injectable Emulsion Placebo into a 5 mL volumetric flask. Add 2.0 mL of Ethanol. Sonicate for 3 minutes with intermittent shaking to dissolve the Clevidipine. Transfer the above solution into Eppendorf vial and centrifuge for 5 minutes at 2500 rpm. Collect the supernatant solution and filter through 0.45μ Nylon syringe filter and then inject.

Sample Solution (0.17 mg/mL of Clevidipine)

Transfer 1.0 mL of Clevidipine Injectable Emulsion Sample into a 5 mL volumetric flask. Add 2.0 mL of Ethanol. Sonicate for 3 minutes with intermittent shaking to dissolve the Clevidipine. Transfer the above solution into Eppendorf vial and centrifuge for 5 minutes at 2500 rpm. Collect the supernatant solution and filter through 0.45μ Nylon syringe filter and then inject.

### Chromatographic Conditions:

TABLE 2
Chromatographic conditions

| Chromatographic Mode | Gradient | |
|---|---|---|
| Column | Inertsil ODS 3 V; 250 mm × 4.6 mm, | |
| | 5 μm, (Supplier: GL | |
| | Sciences, Cat No.: 5020-01802). | |
| Wavelength | 239 | nm |
| Flow rate | 1.0 | mL/min |
| Injection volume | 20 | μL |
| Column oven temperature | 25° | C. |
| Autosampler temperature | 5° | C. |
| Approximate Retention | About 23 minutes | |
| time for Clevidipine | | |
| Run time | 55 | minutes |

*[NOTE-The slower syringe draw rate and higher detector sampling rate can be applied in order to improve the precision.]

### Gradient Program:

TABLE 3
HPLC gradient program

| Time (minutes) | Solution A (%) | Solution B (%) |
|---|---|---|
| 0 | 50 | 50 |
| 5 | 50 | 50 |
| 6 | 54 | 46 |
| 25 | 54 | 46 |
| 30 | 100 | 0 |
| 45 | 100 | 0 |
| 46 | 50 | 50 |
| 55 | 50 | 50 |

### Example 1: Effect of Antioxidants on Stability of Clevidipine in Emulsion

TABLE 4
Compositions of the formulations 40023-18A, 18B and 40023-021

| | 40023-018A | 40023-018B | 40023-021 |
|---|---|---|---|
| Ingredients | Qty/mL | Qty/mL | Qty/mL |
| Clevidipine | 0.5 mg | 0.5 mg | 0.5 mg |
| Egg | 12 mg | 12 mg | 12 mg |
| Phosphatidylcholine | | | |
| (Egg PC) | | | |
| Ethanol | 15.78 mg | 15.78 mg | 15.78 mg |
| Soybean oil | 200 mg | 200 mg | 200 mg |
| Oleic acid | 0.3 mg | 0.3 mg | 0.3 mg |
| Sodium benzoate | 0.83 mg | 0.83 mg | 0.83 mg |
| Methyl paraben | 0.1 mg | 0.1 mg | — |
| α-Tocopherol | 0.5 mg | 0.5 mg | 0.5 mg |
| L-Cysteine free base | NA | 0.1 mg | 0.1 mg |
| Milli-Q-Water | 770 mg | 770 mg | 770 mg |

### Process:

### Preparation of Oil Phase:

Step 1: Egg PC and ethanol were added to a glass vial and sonicated until a clear solution was formed.

Step 2: Tocopherol and Oleic acid were weighed into a 150 mL glass beaker; Soybean oil was added to the beaker and mixed using a magnetic stir bar at 500 RPM for 5 minutes.

Step 3: Clevidipine was added to Step 2 solution, continued mixing at 75° C. until the Clevidipine was completely dissolved (Approximately mixing time: 12 minutes at 75° C.). Solution was then cooled to room temperature.

Step 4: The Egg PC solution from Step 1 was added to Step 3 solution, mixed at 1500 RPM using an overhead stirrer for about 2 minutes and this completes the oil phase preparation.

### Preparation of Water Phase

Step 5, Water and methyl paraben were transferred into a 100 mL glass beaker and sonicated until a clear solution is formed. It took about 7 minutes to dissolve methyl paraben.

Step 6: Sodium Benzoate was added to Step 5 solution and mixed using a magnetic stir bar until a clear solution is formed, and this completes the preparation of the water phase.

### Preparation of Coarse Emulsion

Step 7: Water phase from Step 6 was added to oil phase of Step 4 slowly under continuous stirring using overhead stirrer at 1500 RPM. After completion of addition of water phase to the oil phase, mixing was continued for 10 minutes.

### Preparation of Microemulsion:

Step 8: Coarse emulsion from Step 7 was milled through a microfluidizer at 20K PSI for 6 cycles and 25K PSI for another 6 cycles. Final mean globule size (Z-average) was maintained at 130-160 nm. (Note: Globule size was measured by diluting 1 mL emulsion to 100 mL with water)

### Addition of L-Cysteine:

40023-018A: L-Cysteine not added
40023-018B: L-Cysteine was added to Step-8, nano emulsion and sonicated for 5 minutes to dissolve.
40023-021: L-Cysteine was added to water phase at Step-6.

### Results:

TABLE 5
Results of Stress Stability of the
formulations 40023-18 and 40023-21.

| | 40023-018A | 40023-018B | 40023-021 |
|---|---|---|---|
| | 60° C., | 60° C., | 60° C., |
| Test Parameter | 7 Day | 7 Day | 7 Day |
| Description | White color | White color | White color |
| | emulsion | emulsion | emulsion |
| Assay (%) | 66.5 | 97.1 | 58.9 |
| Organic Impurities | | | |
| (% Area Normalization) | | | |
| Any unspecified impurity | 0.51 | ND | ND |
| (%) (RRT@0.21) | | | |
| Any unspecified impurity | 9.18 | ND | 7.18 |
| (%) (RRT@0.23) | | | |
| Any unspecified impurity | ND | ND | 1.3 |
| (%) (RRT@0.24) | | | |
| Any unspecified impurity | 1.22 | ND | ND |
| (%) (RRT@0.25) | | | |
| Any unspecified impurity | ND | ND | 3.21 |
| (%) (RRT@0.26) | | | |
| Any unspecified impurity | ND | ND | 0.62 |
| (%) (RRT@0.28) | | | |
| Any unspecified impurity | 2.56 | ND | 4.12 |
| (%) (RRT@0.30) | | | |
| Any unspecified impurity | ND | ND | 0.83 |
| (%) (RRT@0.32) | | | |
| Any unspecified impurity | 0.63 | ND | 0.6 |
| (%) (RRT@0.36) | | | |
| Any unspecified impurity | 4.15 | ND | 3.64 |
| (%) (RRT@0.40) | | | |
| Any unspecified impurity | 6.18 | ND | 8.36 |
| (%) (RRT@0.43) | | | |
| Any unspecified impurity | 0.42 | ND | 0.38 |
| (%) (RRT@0.47) | | | |
| Any unspecified impurity | 0.25 | ND | ND |
| (%) (RRT@0.59) | | | |
| Any unspecified impurity | 0.20 | ND | 0.11 |
| (%) (RRT@0.63) | | | |
| Any unspecified impurity | 0.29 | ND | ND |
| (%) (RRT@0.68) | | | |
| Any unspecified impurity | 2.14 | ND | ND |
| (%) (RRT@0.70) | | | |
| Any unspecified impurity | ND | 0.27 | 0.3 |
| (%) (RRT@0.73) | | | |
| Any unspecified impurity | 0.44 | ND | 0.11 |
| (%) (RRT@0.75) | | | |
| Any unspecified impurity | 0.09 | ND | 0.04 |
| (%) (RRT@0.81) | | | |
| Any unspecified impurity | ND | ND | 0.13 |
| (%) (RRT@0.85) | | | |
| Any unspecified impurity | 4.76 | 0.74 | 4.61 |
| (%) (RRT@0.90) | | | |
| Total impurities (%) | 33.02 | 1.01 | 35.54 |

### Conclusion:

The L-Cysteine has significant stabilizing effect on Clevidipine in the emulsion. It reduces impurities to just 1% compared to >30% without Cysteine.

The process of adding cysteine during the manufacturing of emulsion was found to exert surprisingly stabilizing effects if added to nano-emulsion after coarse milling and formation of the microemulsion.

Addition of Cysteine to water phase before the formation of emulsion did not elicit any positive effect on the stability of the Clevidipine in the emulsion.

### Example-2: Effect of L-Cysteine on Physicochemical Properties of Emulsion Autoclaving

The effect of Cystine on autoclaving was evaluated with the following two formulations prepared with and without cysteine.

TABLE 6
Compositions for 40023-028 and 40023-029

| Ingredient | 40023-028 Qty/mL | | 40023-029 Qty/mL | |
|---|---|---|---|---|
| Clevidipine | 0.5 | mg | 0.5 | mg |
| Egg Phosphatidyl choline | 12 | mg | 12 | mg |
| Soybean oil | 200 | mg | 200 | mg |
| Glycerin | 22.5 | mg | 22.5 | mg |
| Oleic Acid | 0.3 | mg | 0.3 | mg |
| α-Tocopherol | 0.0 | mg | 0.5 | mg |
| EDTA-Na | 0.05 | mg | 0.0 | mg |
| L- Cysteine free base | 0.0 | | 0.1 | mg |
| Milli-Q-Water | 764.1 | mg | 764.1 | mg |

### Process:

### Oil Phase Preparation:

Step 1 Add Soybean oil into a heat jacketed mixing vessel. Heat the oil to 80° C. with mixing, Add Clevidipine and continue mixing until Clevidipine is completely dissolved and a clear solution is formed. Allow the solution to cool to room temperature.

Step 2: Add Oleic acid to Step 1 mixture and mix until solution is clear. (Note: Tocopherol was added in this along with oleic acid in this step for the formulation which had tocopherol in the composition)

Step 3: In another small heat jacketed mixing vessel, charge glycerin and heat to 40° C. while mixing. Add Egg Phosphatidylcholine while mixing and maintaining temperature of 40° C. Continue mixing until uniform thick slurry is formed.

### Water Phase Preparation:

### Step-4:

40023-029:

In another mixing tank, charge water and add Methyl paraben and mix until a clear solution is formed.

40023-028

In another mixing tank, charge water and add EDTA and mix until a clear solution is formed.

### Preparation of Coarse Emulsion:

Step 5: Add Step 2 solution to Egg PC dispersion of Step 3. Mix for about 10 minutes to uniform hazy dispersion.

Step 6: Add water phase from step-4 to oil mixture of Step 5 slowly while mixing. Continue mixing for 20 minutes after the addition of the water phase.

### Nano Emulsion Preparation:

Step 7: Mill the coarse emulsion from step 6 by passing through microfluidizer for 8 cycles at 25 PSI. Add L-Cysteine free base and adjust pH to 6.9-7.1 using 1 M NaOH. The pH mentioned here is the value recorded during the in-process check.

### Autoclaving:

The 5 mL of the emulsion was filled in 5 mL Type 1 glass vials. Filled vials were subjected to autoclaving at two different conditions, 115° C. for 20 minutes and 121° C. for 20 minutes.

### Results:

TABLE 7
Stability Results of the Formulations 40023-028 and 40023-029

| | 40023-029 | | 40023-028 | |
|---|---|---|---|---|
| | | 1M-40° C./ | | 1M 14D - |
| | | 75% RH | | 40° C./75% RH |
| | | Autoclave | | Autoclave |
| Test Parameter | Initial | @115° C.-20 min | Initial | @115° C.-20 min |
| Description | White milky | White milky | White milky | White milky |
| | emulsion | emulsion | emulsion | emulsion, but |
| | | | | coarser globules |
| pH (Dilute 1 mL Sample to | 7.561 | 4.619 | 7.639 | 4.143 |
| 10 mL with water) | | | | |
| Globule size (Mean) (nm) | 171.1 | 141.1 | 152 | 1190 |
| (Dilute 1 mL Sample to | | | | |
| 100 mL with water) | | | | |
| Zeta Potential (mv) (Dilute | −33.15 | −43.44 | −31.8 | −28.36 |
| 1 mL Sample to 100 mL with | | | | |
| water) | | | | |
| Assay (%) | 104.5 | 93.4 | 100.4 | 94.8 |

Organic impurities (%)

| | | | | |
|---|---|---|---|---|
| Any unspecified impurity | ND | 0.26 | 0.05 | 1.22 |
| (%) (RRT@0.90) | | | | |
| Total impurities (%) | ND | 0.26 | 0.05 | 1.22 |

### Conclusion:

The formulation 40023-028 is same as RLD composition and does not contain cysteine; after autoclaving it looked like a coarser emulsion. After one month at 40° C./75% RH, it was observed that the emulsion almost phase separated.

The impurity at RRT 0.90 increased to 1.20%.

In contrast, 40023-029 was stable after one month at 40° C./75% RH, there was no phase separation and also total impurities were less than 0.30%.

L-Cysteine plays significant role in chemical and physical stability of emulsion.

### Example-3: Effect of Increasing Concentration of L-Cysteine on the Stability of the Emulsion

Effect of Cysteine on the formulation stability was evaluated with the following 3 formulations containing cysteine at 0.5 mg/mL, 1.0 mg/mL and 1.5 mg/mL concentrations:

TABLE 8
Compositions for Formulations
40023-044A, 044B and 40023-044C

| | 40023-044A | | 40023-044B | | 40023-044C | |
|---|---|---|---|---|---|---|
| Ingredient | Qty/mL | | Qty/mL | | Qty/mL | |
| Clevidipine | 0.5 | mg | 0.5 | mg | 0.5 | mg |
| Egg Phosphatidyl choline | 14 | mg | 14 | mg | 14 | mg |
| Soybean oil | 200 | mg | 200 | mg | 200 | mg |
| Glycerin | 22.5 | mg | 22.5 | mg | 22.5 | mg |
| Oleic Acid | 0.3 | mg | 0.3 | mg | 0.3 | mg |
| α-Tocopherol | 0.2 | mg | 0.2 | mg | 0.2 | mg |
| Methyl Paraben | 0.1 | mg | 0.1 | mg | 0.1 | mg |
| L- Cysteine free base | 0.5 | mg | 1.0 | mg | 1.5 | mg |
| Milli-Q-Water | 764.1 | mg | 764.1 | mg | 764.1 | mg |

### Process:

### Oil Phase Preparation:

Step 1: In the heat jacketed mixing vessel, charge Soyabean oil. Heat the oil to 80° C. with mixing, add Clevidipine and continue mixing until all of the Clevidipine is dissolved, and a clear solution is formed. Allow the solution to cool to room temperature.

Step 2: Add Oleic acid to Step 1 mixture and mix until solution is clear. (Note: Tocopherol was added along with oleic acid in this step for the formulation which had tocopherol in the composition)

Step 3: In another small heat jacketed mixing vessel, charge glycerin and heat to 40° C. while mixing. Add Egg Phosphatidylcholine while mixing and maintaining temperature of 40° C. Continue mixing until uniform thick slurry is formed.

### Water Phase Preparation:

Step 4: In another mixing tank, charge water and add Methyl paraben and mix until a clear solution is formed.

### Preparation of Coarse Emulsion:

Step 5: Add Step 2 solution to Egg PC dispersion of Step 3. Mix for about 10 minutes to uniform hazy dispersion.

Step 6: Add water phase from Step 4 to the oil mixture from Step 5 slowly while mixing. Continue mixing for 20 minutes after the addition of the water phase.

### Nano Emulsion Preparation:

Step 7: Mill the coarse emulsion from Step 6 by passing through microfluidizer for 8 cycles at 25 PSI. Add L-Cysteine free base and adjust pH to 6.9-7.1 using 1 M NaOH. The pH mentioned here is the value recorded during in-process check.

### Autoclaving:

The 20 mL of the emulsion was filled in Type 1, 20 mL glass vials. Filled vials were subjected to autoclaving at 121° C. for 20 minutes.

### Results:

TABLE 9A
Stability data of Formulations 40023-44A

| | 40023-044A | | |
|---|---|---|---|
| | Initial | | |
| | Autoclave@ | 1M-40° C./ | 2M-40° C./ |
| Test Parameter | 121° C.-20 min | 75% RH | 75% RH |
| Description | White milky | White milky | White milky |
| | emulsion | emulsion | emulsion |
| pH (Dilute 1 mL Sample | 6.794 | 6.44 | 6.141 |
| to 10 mL with water) | | | |
| Globule size (Mean) | 222.8 | 515.8 | 398 |
| (nm) (Dilute 1 mL | | | |
| Sample to 100 mL with | | | |
| water) | | | |
| Zeta Potential (mv) | −26.86 | −41.09 | −43.02 |
| (Dilute 1 mL Sample | | | |
| to 100 mL with water) | | | |
| Assay (%) | 103.5 | 105.0 | 106.8 |
| Phosphatidylcholine | 111.9 | 104.9 | 104.8 |
| PC (%) | | | |
| Lysophosphatidyl- | 0.0 | 0.8 | 3.4 |
| choline LPC (%) | | | |

Organic Impurities (% Area Normalization)

| | | | |
|---|---|---|---|
| Any unspecified | 0.13 | 0.14 | 0.20 |
| impurity (%) | | | |
| (RRT@0.90) | | | |
| Total impurities (%) | 0.13 | 0.14 | 0.20 |

TABLE 9B
Stability data of Formulations 40023-044B

| | 40023-044B | | |
|---|---|---|---|
| | Initial | | |
| | Autoclave@ | 1M-40° C./ | 2M-40° C./ |
| Test Parameter | 121° C.-20 min | 75% RH | 75% RH |
| Description | White milky | White milky | White milky |
| | emulsion | emulsion | emulsion |
| pH (Dilute 1 mL Sample | 6.474 | 6.248 | 6.052 |
| to 10 mL with water) | | | |
| Globule size (Mean) | 325.5 | 537.2 | 419.6 |
| (nm) (Dilute 1 mL | | | |
| Sample to 100 mL with | | | |
| water) | | | |
| Zeta Potential (mv) | −26.85 | −33.77 | −36.79 |
| (Dilute 1 mL Sample | | | |
| to 100 mL with water) | | | |
| Assay (%) | 104.5 | 104.6 | 104.8 |
| Phosphatidylcholine | 112.3 | 102.3 | 99.2 |
| PC (%) | | | |
| Lysophosphatidyl- | 0.0 | 0.9 | 7.9 |
| choline LPC (%) | | | |

Organic Impurities (% Area Normalization)

| | | | |
|---|---|---|---|
| Any unspecified | 0.13 | 0.28 | 0.44 |
| impurity (%) (RRT@0.90) | | | |
| Total impurities (%) | 0.13 | 0.28 | 0.44 |

TABLE 9C
Stability data of Formulations 40023 -044C

| | 40023-044C | | |
|---|---|---|---|
| | Initial | | |
| | Autoclave@ | 1M-40° C./ | 2M-40° C./ |
| Test Parameter | 121° C.-20 min | 75% RH | 75% RH |
| Description | White milky | White milky | White milky |
| | emulsion | emulsion | emulsion |
| pH (Dilute 1 mL Sample | 5.871 | 6.219 | 5.912 |
| to 10 mL with water) | | | |
| Globule size (Mean) | 616.5 | — | 428 |
| (nm) (Dilute 1 mL | | | |
| Sample to 100 mL with | | | |
| water) | | | |
| Zeta Potential (mv) | −43.12 | — | −40.21 |
| (Dilute 1 mL Sample | | | |
| to 100 mL with water) | | | |
| Assay (%) | 102.6 | 103.9 | 104.5 |
| Phosphatidylcholine | 111.1 | 87.9 | 95.3 |
| PC (%) | | | |
| Lysophosphatidyl- | 0.0 | 1.5 | 9.6 |
| choline LPC (%) | | | |

Organic Impurities (% Area Normalization)

| | | | |
|---|---|---|---|
| Any unspecified | 0.18 | 0.47 | 0.53 |
| impurity (%) (RRT@0.90) | | | |
| Total impurities (%) | 0.18 | 0.47 | 0.53 |

### Conclusion:

The results indicate that the amount of L-Cysteine must be <0.5 mg/mL. Otherwise increase in L-Cysteine quantity in the formulations leads to increase in impurities, especially one eluting at RRT 0.9. The results also show the decomposition of Phosphatidylcholine and increase in Lysophosphatidylcholine with increase in concentration of cysteine n the formulation.

The L-Cysteine at 0.1 mg/mL shows similar stabilizing effect as that of 0.5 mg/mL. Results are discussed below.

### Example-4: Effect of Autoclaving Temperature on Physicochemical Characteristics and Stability of Clevidipine Emulsion

TABLE 10
Composition for 40023-042

| | 40023-042 | |
|---|---|---|
| Ingredient | Qty/mL | |
| Clevidipine | 0.5 | mg |
| Egg Phosphatidyl choline | 14 | mg |
| Soyabean oil | 200 | mg |
| Glycerin | 22.5 | mg |
| Oleic Acid | 0.3 | mg |
| Methyl Paraben | 0.1 | mg |
| L- Cysteine free base | 0.1 | mg |
| Milli-Q-Water | 764.1 | mg |

### Process:

Same as that was followed for Formulation 40023-044 but autoclaved at 121° C./20 minutes and 115° C./20 minutes.

### Results:

TABLE 11A
Data on effect of Autoclaving Temperature on Physicochemical
Properties and Stability 40023-042A

| | 40023-042A | | | |
|---|---|---|---|---|
| | | 1M-40° C./ | 2M-40° C./ | 3M- 40° C./ |
| | Initial | 75% RH | 75% RH | 75% RH |
| | Autoclave@ | Autoclave @ | Autoclave @ | Autoclave @ |
| Parameters | 115° C.-20 min | 115° C.-20 min | 115° C.-20 min | 115° C. -20 min |
| Description | White milky | White milky | White milky | White milky |
| | emulsion | emulsion | emulsion | emulsion |
| pH (Dilute 1. mL Sample | 7.068 | 5.807 | 5.635 | 6.054 |
| to 10 mL with water) | | | | |
| Globule size (Mean) (nm) | 144.2 | 148.4 | 162.3 | 156.4 |
| (Dilute 1 mL Sample to | | | | |
| 100 mL with water) | | | | |
| Zeta Potential (mv) | −32.15 | −33.22 | −34.45 | −35.99 |
| (Dilute 1 mL Sample to | | | | |
| 100 mL with water) | | | | |
| Assay (%) | 102.0 | 102.6 | 104.0 | 97.4 |
| PC (%) | 108.4 | 97.9 | 103.0 | 82.3 |
| LPC (%) | 0.0 | 0.0 | 5.4 | 19.9 |
| | Organic Impurities (% Area Normalization) | | | |
| Any unspecified impurity | ND | 0.16 | 0.21 | 0.31 |
| (%) (RRT@0.90) | | | | |
| Total impurities (%) | ND | 0.16 | 0.21 | 0.31 |

TABLE 11B
Data on effect of Autoclaving Temperature on Physicochemical
Properties and Stability 40023-042B

| | 40023-042B | | | |
|---|---|---|---|---|
| | Initial | 1M-40° C./ | 2M-40° C./ | 3M-40° C./ |
| | Autoclave | 75% RH | 75% RH | 75% RH |
| | @121° | Autoclave @ | Autoclave @ | Autoclave @121° |
| Parameters | C.- 20 min | 121° C.- 20 min | 121° C.- 20 min | C. -20 min |
| Description | White milky | White milky | White milky | White milky |
| | emulsion | emulsion | emulsion | emulsion |
| pH (Dilute 1. mL Sample | 6.958 | 6.149 | 5.645 | 5.830 |
| to 10 mL with water) | | | | |
| Globule size (Mean) (nm) | 401.9 | 315 | 287.2 | 325.4 |
| (Dilute 1 mL Sample to | | | | |
| 100 mL with water) | | | | |
| Zeta Potential (mv) | −30.7 | −34.5 | −38.83 | −36.35 |
| (Dilute 1 mL Sample to | | | | |
| 100 mL with water) | | | | |
| Assay (%) | 102.5 | 104.4 | 103.8 | 97.7 |
| PC (%) | 104.0 | 100.0 | 103.8 | 89.9 |
| LPC (%) | 0.0 | 0.0 | 5.3 | 14.1 |
| | Organic Impurities (% Area Normalization) | | | |
| Any unspecified impurity | 0 | 0.19 | 0.23 | 0.28 |
| (%) (RRT@0.90) | | | | |
| Total impurities (%) | 0 | 0.19 | 0.23 | 0.28 |

### Conclusion:

Data indicates that the after autoclaving at 121° C. for 20 minutes results in increase in the globule size of the emulsion.

Chemical stability remains same for vials autoclaved at both 115° C. for 20 minutes and 121° C. for 20 minutes.

The autoclaving condition 115° C. for 20 minutes is desirable if an autoclave with no rotary option is available in the autoclave.

The autoclaving condition 121° C. for 20 minutes may require a rotary autoclave to arrest increase in the globule size during the autoclaving.

### Example-5: Effect of Fill Volume on Globule Size During Autoclaving at 121° C.

Small volume batches were made and filled with small volumes and the effect of volume on the globule size during autoclaving at 121° C. for 20 minutes was evaluated.

TABLE 12
Composition for Testing Effect Volume on Globule Size

| Ingredient | 40023-033 Qty/mL | | 40023-042 Qty/mL | | 40023-048 Qty/mL | |
|---|---|---|---|---|---|---|
| Clevidipine | 0.5 | mg | 0.5 | mg | 0.5 | mg |
| Egg Phosphatidyl choline | 14 | mg | 14 | mg | 14 | mg |
| Soyabean oil | 200 | mg | 200 | mg | 200 | mg |
| Glycerin | 22.5 | mg | 22.5 | mg | 22.5 | mg |
| Oleic Acid | 0.3 | mg | 0.3 | mg | 0.3 | mg |
| Methyl Paraben | 0.1 | mg | 0.1 | mg | 0.1 | mg |
| L- Cysteine free base | 0.1 | mg | 0.1 | mg | 0.1 | mg |
| Milli-Q-Water | 764.1 | mg | 764.1 | mg | 764.1 | mg |

### Process:

Same process described for 40023-44 was followed, but volume filled in the vial for autoclaving is different as mentioned below.

TABLE 13
Fill volume details for autoclaving

| Batch. Number of the Formulation | Fill Volume for Autoclaving |
|---|---|
| 4003-033 | 5 mL in 5 mL vial |
| 40023-42 | 18 mL in 20 mL vial |
| 40023-48 | 50 mL in 50 mL vial |

These vials were autoclaved at 121° C. for 20 minutes.

### Results:

TABLE 14
Results of Autoclaving with Different Volumes of Emulsion

| Parameters | 40023-33 | 40023-42 | 40023-48 |
|---|---|---|---|
| Fill Volume | 5 mL in | 18 mL in | 50 mL in |
| | 5 mL vial | 20 mL vial | 50 mL vial |
| Initial pH before autoclaving | 7.17 | 6.961 | 6.904 |
| Mean Globule Size (nm) after | 226 | 401.9 | 878 |
| autoclaving | | | |

### Conclusion:

The globule size was found to increase as the volume of emulsion autoclaved increased. This is true for autoclaving at 121° C./20 minutes.

Either a rotary autoclave or fine tuning of the formulation is needed to address the issue of increase in globule size during autoclaving.

Alternatively, at 115° C./20 minutes can be adopted.

### Example 6: Effect of pH on Globule Size During Autoclaving

The previous trials revealed that autoclaving at 115° C. for 20 minutes did not result in the changes in the mean globule size of the emulsion. However, the autoclaving cycle, 121° C. for 20 minutes resulted in increased globule size. In this experiment, the effect of pH was evaluated to stabilize the globule size during autoclaving at 121° C.

TABLE 15
Composition for Testing Effect of pH
on Globule Size During Autoclaving

| | 40023-046/40023-053 | |
|---|---|---|
| Ingredients | Qty/mL | |
| Clevidipine | 0.5 | mg |
| Glycerine | 22.5 | mg |
| Egg Phosphatidylcholine | 14 | mg |
| Soybean oil | 200 | mg |
| Oleic acid | 0.3 | mg |
| Methylparaben | 0.08 | mg |
| L-Cysteine free base | 0.1 | mg |
| Milli-Q-Water | 764.1 | mg |

### Process:

Same as 40023-044 but after milling emulsion through microfluidizer, emulsion was divided into two lots and different pH was adjusted for each lot. After pH adjustment, emulsions were filled in Type 1 glass vials, 50 mL in 50 mL vial, closed with ETFE rubber stopper and Aluminum flip-off seals. A third batch 40023-046 was also used in this study and three lots were adjusted to different pH levels as shown in Table 16. Vials from these three batches were autoclaved at 121° C. for 20 minutes.

TABLE 16
pH value of 40023-053 before autoclaving

| Formulation | pH values before autoclaving |
|---|---|
| 40023-046 | 6.958 |
| 40023-053B | 7.21 |
| 40023-053C | 7.539 |

### Results:

TABLE 17
Results of Effect of pH on Globule Size after Autoclaving

| | | Mean Globule Size | Mean Globule Size |
|---|---|---|---|
| | pH Adjusted | After Autoclaving | After Autoclaving |
| | to (Before | at 121° C. | at 115° C. for |
| Formulation | Autoclaving) | for 20 minutes | 20 minutes |
| 40023-046 | 6.95 | 370.9 nm | Not Done |
| 40023-053B | 7.21 | 245.6 nm | 156.4 nm |
| 40023-053C | 7.53 | 148.6 nm | Not Done |

Note:
Initial mean globule size before Autoclaving was maintained at 145-160 nm

### Conclusion:

The formulation pH must be >7.50 before autoclaving to maintain globule size during autoclaving at 121° C. for 20 minutes.

Rotary autoclave is necessary if lower pH (<7.5) is required for stability reasons.

Alternatively, low temperature autoclave cycle is necessary, (115° C./20 minutes) to retain globule size after autoclaving without opting for higher pH.

### Example-7: Clevidipine Emulsion with Sodium Benzoate as Preservative

In the following trials, amount of sodium benzoate, pH of the formulation and fill volume were tested.

TABLE 18
Compositions of Clevidipine emulsion
with sodium benzoate as preservative

| | 40023-049 | | 40023-052 | | 40023-054 | | 40023-055 | |
|---|---|---|---|---|---|---|---|---|
| Ingredients | Qty/mL | | Qty/mL | | Qty/mL | | Qty/mL | |
| Clevidipine | 0.5 | mg | 0.5 | mg | 0.5 | mg | 0.5 | mg |
| Glycerine | 22.5 | mg | 22.5 | mg | 22.5 | mg | 22.5 | mg |
| Egg PC | 14 | mg | 14 | mg | 14 | mg | 14 | mg |
| Soybean oil | 200 | mg | 200 | mg | 200 | mg | 200 | mg |
| Oleic acid | 0.3 | mg | 0.3 | mg | 0.3 | mg | 0.3 | mg |
| Sodium Benzoate | 0.1 | mg | 0.5 | mg | 0.1 | mg | 0.1 | mg |
| L-Cysteine free base | 0.1 | mg | 0.1 | mg | 0.1 | mg | 0.1 | mg |
| Milli-Q-Water | 764.1 | mg | 764.1 | mg | 764.1 | mg | 764.1 | mg |

### Process:

### Oil Phase Preparation:

Step 1: In the heat jacketed mixing vessel, charge Soyabean oil. Heat the oil to 80° C. with mixing, add Clevidipine and continue mixing until all of the Clevidipine is dissolved and a clear solution is formed. Allow the solution to cool to room temperature.

Step 2: Add Oleic acid to Step 1 mixture and mix until solution is clear.

Step 3: In another small heat jacketed mixing vessel, charge glycerin and heat to 40° C. while mixing. Add Egg Phosphatidylcholine while mixing and maintaining temperature at 40° C. Continue mixing until uniform thick slurry is formed.

### Water Phase Preparation:

Step 4: In another mixing tank, charge water and add sodium benzoate and mix until a clear solution is formed.

### Preparation of Coarse Emulsion:

Step 5: Add step-2 solution to Egg PC dispersion of Step 3. Mix for about 10 minutes to uniform hazy dispersion.

Step 6: Add water phase from step-4 to oil mixture of Step 5 slowly while mixing. Continue mixing for 20 minutes after the addition of the water phase.

### Nano Emulsion Preparation:

Step 7: Mill the coarse emulsion from Step 6 by passing through a microfluidizer for 8 cycles at 25 PSI. Add L-Cysteine free base and adjust pH to 6.9-7.5 using 1 M NaOH. The pH mentioned here is the value recorded during in-process check.

### Autoclaving:

40023-049-Filled 18 mL in 20 mL vial; 40023-052-Filled 18 mL in 20 mL vial; 40023-054-Filled 5 mL in 10 mL vial; 40023-055-Filled 50 mL in 50 mL vial Filled vials were subjected to autoclaving at 121° C. for 20 minutes.

### Results: Effect of Sodium Benzoate Concentration on Physical Stability of Emulsion During Autoclaving

TABLE 19
Effect of Sodium Benzoate concentration of Physical Stability of Emulsion

| | Sodium | | Mean Globule Size | |
|---|---|---|---|---|
| | Benzoate | pH of | after autoclaving | |
| | concentration | coarse | at 121° C. for 20 | |
| Formulations | (Mg/mL) | emulsion | minutes | Conclusion |
| 40023-049 | 0.1 | 6.99 | 824 | Sodium Benzoate must |
| | | | | be 0.1 mg/mL or less |
| 40023-052 | 0.5 | 6.88 | Phase separation observed | for physical stability |
| | | | | of emulsion |

Effect of pH and Fill volume on Globule Size during autoclaving

TABLE 20
Data on pH Effect on Globule Size of Emulsion after Autoclaving

| | | | Mean globule size | | |
|---|---|---|---|---|---|
| | pH of emulsion | | after autoclaving | | |
| Formulation | before autoclaving | Fill volume | (121° C./20 minutes) | | Conclusion |
| 40023-054 | 7.537 | 5 ml in | 189 | nm | pH of >7.5 is needed |
| | | 10 mL vial | | | to keep globule size |
| | | | | | unchanged after |
| 40023-055A | 7.023 | 50 ml in | 800.8 | nm | autoclaving at |
| | | 50 mL vial | | | 121° C./20 minutes. |
| | | | | | Otherwise, rotary |
| 40023-055B | 7.206 | 50 ml in | 350.9 | nm | autoclave or low |
| | | 50 mL vial | | | temperature cycle, |
| 40023-055C | 7.53 | 50 ml in | 148.1 | nm | 115° C./20 minutes is |

| | |
|---|---|
| 50 mL vial | necessary. |

TABLE 21
Stability data of Clevidipine Emulsion
with Sodium Benzoate as Preservative

| | 40023-054 | |
|---|---|---|
| | Initial | 1M-40° C./75% RH |
| | Autoclave@121° | Autoclave@121° |
| Test Parameter | C.-20 min | C.-20 min |
| Description | White milky | White milky |
| | emulsion | emulsion |
| pH (Dilute 1 mL Sample | 7.406 | 7.138 |
| to 10 mL with water) | | |
| Globule size (Mean) (nm) | 189 | 182.37 |
| (Dilute 1 mL Sample to | | |
| 100 mL with water) | | |
| Zeta Potential (mv) | −14.47 | −17.65 |
| (Dilute 1 mL Sample | | |
| to 100 mL with water) | | |
| Assay (%) | 94.9 | 92.0 |

Organic Impurities (% Area Normalization)

| | | |
|---|---|---|
| Any unspecified impurity | 0.04 | 0.18 |
| (%) (RRT@0.90) | | |
| Total impurities | 0.04 | 0.18 |
| (%)[Reporting threshold | | |
| 0.1% not applied | | |
| in the summation] | | |

### Conclusion:

Sodium Benzoate at 0.1 mg/mL is optimum and higher concentrations leads to phase separation.

In order to keep globule size largely unchanged during autoclaving, pH of the emulsion must be 7.5 or more.

If the pH of emulsion between 7 to 7.25 is desired, rotary autoclave or low temperature 115° C./20 minutes are necessary.

The formulation 40023-054 with 0.1 mg/mL of Sodium Benzoate and pH 7.5 found to be stable at 40° C./75% for at least one month.

### Example-8: Adventitious Microbial Contamination Testing of Selected Clevidipine Formulations

TABLE 22
Compositions tested for Adventitious Contamination.

| | Batch No.: | | | Cleviprex ® |
|---|---|---|---|---|
| | 40023-058 | Batch No.: | Batch No.: | Batch No.: |
| | Manufacturing | 40023-059 | 40023-060 | 1173362 |
| | Date: 08/2024 | Manufacturing | Manufacturing | Expiry Date: 08/2024 |
| Ingredient | Quantity: mg/mL | Date: 08/2024 | Date: 08/2024 | Quantity: mg/mL |
| Clevidipine | 0.5 | 0.5 | 0.5 | 0.5 |
| Purified egg yolk | 14 | 14 | 14 | 12 |
| phospholipids | | | | |
| Soybean oil | 200 | 200 | 200 | 200 |
| Glycerin | 22.5 | 22.5 | 22.5 | 22.5 |
| Oleic acid | 0.3 | 0.3 | 0.3 | 0.3 |
| Sodium Benzoate | — | 0.1 | — | — |
| Methylparaben | 0.05 | — | 0.08 | — |
| L- Cysteine free | 0.1 | 0.1 | 0.1 | — |
| Disodium | — | — | — | 0.05 |
| Water for Injection | 764.1 mg | 764.1 mg | 764.1 mg | Q s to 1 mL |

### Procedure for Testing Adventitious Contamination Testing

Take the required quantity of vials of Clevidipine Injection USP, 0.5 mg/mL.

Take separate vials of Clevidipine Injection for each Microorganisms mentioned in the Table-32.

The concentration of the suspension inoculum to be used in the test sample of Clevidipine Injection-0.5 mg/mL was adjusted to about 50 cfu/mL.

Inoculate each container of Clevidipine Injection USP, 0.5 mg/mL with test Microorganism mentioned in Table-23. The concentration of each test Microorganisms in each container after inoculation is NMT 100 cfu/50 mL. The concentration of inoculum after inoculation should not be more than 4% of the total volume of the sample. For a 50 mL vial, 2 mL of microbial suspension was added through syringe and needle via needle puncturing of rubber stopper. Similarly 0.8 mL microbial suspension was transferred to 20 ml vials.

At 0, 24 and 48 hours interval, a 10 mL of the sample was withdrawn from each container and filtered through a membrane filtration unit containing 0.45 μm×47 mm membrane filter.

The filter membrane was rinsed to remove preservatives with the validated rinses. The membrane was placed into a plate and appropriate medium was poured as per Tables 22 and 23. The plates were incubated plates as per Table 23 and count the number of colonies after incubation and report results.

TABLE 22
Details of Microbial Inoculation of Clevidipine Injection

| | Inoculation organism | | |
|---|---|---|---|
| | and volume to be | Storage temp (° C.) | |
| Product | injected to each vial | for vials after inoculation | Sampling time points |
| 40023-058 | Inject 0.8 mL | 20-25° C. | 0, 12, 24 and 48 hours. |
| First set of three | suspension/culture of | | Take out 10 mL product |
| vials | Escherichia coli ATCC | | via sterile needle-syringe. |
| | No. 8739 suspension to | | Filter through the |
| | each vial | | membrane filtration unit |
| 40023-058 | Inject 0.8 mL | | containing 0.45 μm × 47 |
| second set of three | suspension/culture of | | mm membrane filter. |
| vials | Pseudomonas aeruginosa | | Incubate whole filter into |
| | ATCC No. 9027 to each | | a plate with media. |
| | vial | | |
| 40023-058 | Inject 0.8 mL | | 0, 12, 24 and 48 hours. |
| Third set of three | suspension/culture of of | | Take out 10 mL product |
| vials | Candida albicans ATCC | | via sterile needle-syringe. |
| | No. 10231 and | | Filter through the |
| | 0.8 mL of Aspergillus | | membrane filtration unit |
| | Niger | | containing 0.45 μm × 47 |
| | ATCC No. 16404 to each | | mm membrane filter. |
| | vial. | | Incubate whole filter into |
| | | | a plate with media for |
| | | | both Candida albicans |
| | | | Aspergillus Niger. |

RLD Injection Details

| | | | |
|---|---|---|---|
| Cleviprex ® | Inject 2 mL | 20-25° C. | 0, 12, 24 and 48 hours. |
| Injection | suspension/culture of | | Take out 10 mL product |
| First vial | Escherichia coli ATCC | | via sterile needle-syringe. |
| | No. 8739 suspension to | | Filter through the |
| | each vial. | | membrane filtration unit |
| | | | containing 0.45 μm × 47 |
| | | | mm membrane filter. |
| | | | Incubate whole filter into |
| | | | a plate with media. |
| Cleviprex ® | Inject 2 mL | 20-25° C. | 0, 12, 24 and 48 hours. |
| Injection | suspension/culture of | | Take out 10 mL product |
| Second vial | Pseudomonas aeruginosa | | via sterile needle-syringe. |
| | ATCC No. 9027 to each | | Filter through the |
| | vial | | membrane filtration unit |
| | | | containing 0.45 μm × 47 |
| | | | mm membrane filter. |
| | | | Incubate whole filter into |
| | | | a plate with media. |
| Cleviprex ® | Inject 2 mL | 20-25° C. | 0, 12, 24 and 48 hours. |
| Injection | suspension/culture of of | | Take out 10 mL product |
| Third vial | Candida albicans ATCC | | via sterile needle-syringe. |
| | No. 10231 and | | Filter through the |
| | 2 mL of Aspergillus Niger | | membrane filtration unit |
| | ATCC No. 16404 to each | | containing 0.45 μm × 47 |
| | vial. | | mm membrane filter. |
| | | | Incubate whole filter into |
| | | | a plate with media for |
| | | | both Candida albicans |
| | | | Aspergillus Niger. |

TABLE 23
Plating and Media details of Incubation Growth of Microbes

| | Suggested | | Inoculum | |
|---|---|---|---|---|
| | Growth | Incubation | incubation | |
| Microorganisms | medium | temperature (° C.) | time | |
| Escherichia coli | SCDM/ | 30-35° C. | 18-24 | hrs |
| ATCC No. 8739 | SCDA | | | |
| Pseudomonas aeruginosa | SCDM/ | 30-35° C. | 18-24 | hrs |
| ATCC No. 9027 | SCDA | | | |
| Candida albicans | SCDM/ | 20-25° C. | 48-72 | hrs |
| ATCC No. 10231 | SDA | | | |
| Aspergillus Niger | SCDM/ | 20-25° C. | 5-7 | days |

| | |
|---|---|
| ATCC No. 16404 | SDA |

### Results

TABLE 24
Results from the Adventitious Contamination Testing

| | Batch | Inoculation | Sampling Time intervals (hrs.) | | | |
|---|---|---|---|---|---|---|
| Product | No. | Organism | 0 | 12 | 24 | 48 |
| Cleviprex, | 1173362 | E. Coli | Nil | Nil | 35 | 106 |
| RLD | | P. Aeruginosa | Nil | Nil | 12 | 50 |
| | | C. Albicans & | Nil | Nil | 41 | 76 |
| | | A. Niger | | | | |
| Clevidipine | 40023-058 | E. Coli | Nil | Nil | Nil | 77 |
| Injectable | | P. Aeruginosa | Nil | Nil | 9 | 41 |
| Emulsion | | C. Albicans & | Nil | Nil | 26 | 62 |
| | | A. Niger | | | | |
| | 40023-059 | E. Coli | Nil | Nil | 12 | 81 |
| | | P. Aeruginosa | Nil | Nil | 8 | 32 |
| | | C. Albicans & | Nil | Nil | 38 | 68 |
| | | A. Niger | | | | |
| | 40023-060 | E. Coli | Nil | Nil | Nil | 58 |
| | | P. Aeruginosa | Nil | Nil | 9 | 26 |
| | | C. Albicans & | Nil | Nil | 29 | 57 |
| | | A. Niger | | | | |

### Conclusion:

The Cleviprex® and Clevidipine Injectable Emulsions of the invention showed similar growth pattern of microorganisms after inoculation and incubation at specific time points.

Number of colonies observed for Cleviprex® and Clevidipine Injectable Emulsions of the invention (40023-058, 59 and 40023-060) were comparable.

### Example-9: Clevidipine Formulations with Enhanced Stability

TABLE 25
Clevidipine compositions with enhanced stability

| | Batch No. 40023-041 | Batch No. 40023-043 |
|---|---|---|
| Ingredient | Qty: mg/mL | Qty: mg/mL |
| Clevidipine | 0.5 | 0.5 |
| Egg phosphatidylcholine | 14 | 12 |
| Soybean oil | 200 | 200 |
| Glycerin | 22.5 | 22.5 |
| α-Tocopherol | 0.2 | 0.5 |
| Oleic Acid | 0.3 | 0.3 |
| Methylparaben | 0.1 | 0.1 |
| L- Cysteine free base | 0.1 | 0.1 |
| Milli-Q-Water | 764.1 | 764.1 |

### Process: Same as 40023-042

TABLE 26A
Stability data of the formulations 40023-041

| | 40023-041 (Autoclave@121° C.-20 min) | | | | |
|---|---|---|---|---|---|
| | Initial after | 3M-40° C./ | 6M-40° C./ | 3M-30° C./ | 6M-30° C./ |
| Test Parameter | autoclaving | 75% RH | 75% RH | 65% RH | 65% RH |
| Description | White milky | White milky | White milky | White milky | White milky |
| | emulsion | emulsion | emulsion | emulsion | emulsion |
| pH | 7.068 | 7.195 | 6.341 | 7.805 | 6.918 |
| Osmolality (mOsm/Kg) | — | — | 334 | — | 329 |
| Globule size (Mean) nm | 233.6 | 279.7 | 293.6 | 283.5 | 299.5 |
| Zeta Potential (mv) | −33.63 | −27.51 | −31.6 | −29.57 | −25.49 |
| Assay (%) | 104.6 | 101.1 | 99.0 | 100.1 | 104.8 |
| PC (%) | 108.5 | 100.4 | 72.4 | 105.5 | 91.0 |
| LPC (%) | 0.0 | 5.4 | 8.7 | 2.3 | 2.7 |

Organic Impurities (% Area Normalization)

| | | | | | |
|---|---|---|---|---|---|
| Any unspecified impurity | ND | 0.16 | 0.21 | 0.11 | 0.09 |
| (%) (RRT@0.90) | | | | | |
| Total impurities (%) | 0.00 | 0.16 | 0.21 | 0.11 | 0.09 |

TABLE 26B
Stability data of the formulations 40023-043

| | 40023-043 (Autoclave@121° C.-20 min) | | | | |
|---|---|---|---|---|---|
| | Initial After | 3M-40° C./ | 6M-40° C./ | 3M-30° C./ | 6M-30° C./ |
| Test Parameter | autoclaving | 75% RH | 75% RH | 65% RH | 65% RH |
| Description | White milky | White milky | White milky | White milky | White milky |
| | emulsion | emulsion | emulsion | emulsion | emulsion |
| pH | 7.453 | 6.792 | 6.309 | 6.514 | 6.920 |
| Osmolality (mOsm/Kg) | — | — | 323 | — | 320 |
| Globule size (Mean) nm | 299.6 | 367.3 | 389.3 | 354.9 | 354.9 |
| Zeta Potential (mv) | −25.45 | −33.06 | −24.17 | −28.97 | −36.98 |
| Assay (%) | 104.2 | 98.9 | 97.4 | 99.8 | 102.7 |
| PC (%) | 101.0 | 94.8 | 62.9 | 104.6 | 86.3 |
| LPC (%) | 0.0 | 8.1 | 13.1 | 2.3 | 2.1 |

Organic Impurities (% Area Normalization)

| | | | | | |
|---|---|---|---|---|---|
| Any unspecified impurity | 0.07 | 0.14 | 0.20 | 0.13 | 0.11 |
| (%) (RRT@0.90) | | | | | |
| Total impurities (%) | 0.07 | 0.14 | 0.20 | 0.13 | 0.11 |

Conclusion from Stability Data:
Both formulations, 40023-041 and 40023-043 were found to be stable at room temperature based on data for 6 months under accelerated stability conditions of 40 C/75% RH.
The concentration of tocopherol at 0.2 mg/mL is adequate.
Hence, a formulation similar to 40023-043 but with reduced tocopherol at 0.2 mg/mL is best suited for a room temperature stable product. Instead of Methyl Paraben, Sodium Benzoate can be used as preservative.
The globule size remained higher than the RLD initially and during stability. To retain original globule size, either autoclaving needs to be done at 1150 C or rotary/water cascade autoclave needs to be used. Another option is to raise the pH to 7.5.
The modified version of formulation 40023-043 with tocopherol at 0.2 mg/mL is also room temperature stable product.

### Example-10: Effect of Cysteine Physicochemical Properties of Clevidipine Emulsion

### Buffering Effect of Cysteine:

Formulations with and without Cysteine were prepared and evaluated for stability of pH before and after autoclaving.

TABLE 27
Clevidipine Emulsion Composition with and without Cysteine

| Ingredient | 40023-086A Qty/mL | | 40023-086B Qty/mL | |
|---|---|---|---|---|
| Clevidipine | 0.5 | mg | 0.5 | mg |
| Egg Phosphatidylcholine | 12 | mg | 12 | mg |
| Soybean oil | 200 | mg | 200 | mg |
| Glycerin | 22.5 | mg | 22.5 | mg |
| Oleic acid | 0.3 | mg | 0.3 | mg |
| Methyl paraben | 0.08 | mg | 0.08 | mg |
| Alpha Tocopherol | 0.2 | mg | 0.2 | mg |
| L-cysteine HCL | 0.1 | mg | 0.0 | |
| Milli-Q-Water | 764.1 | mg | 764.1 | mg |

Process: Same as 40023-042, but dispersion of egg phosphatidylcholine in glycerin was mixed with water with methyl paraben before oil and water phases were mixed to form coarse emulsion.

### Results: Given in Table-28

TABLE 28
Effect of Cysteine on pH during Autoclaving.

| | | | Difference in pH |
|---|---|---|---|
| | pH before | pH after | before and after |
| Formulation | autoclaving | autoclaving | autoclaving |
| 40023-086A | 7.53 | 7.26 | 0.27 |
| (With Cysteine) | | | |
| 40023-086B | 7.74 | 7.15 | 0.60 |
| (With No Cysteine) | | | |

### Conclusion:

Cysteine shows buffering effect and helps in resisting pH change during the autoclaving. Formulation with cysteine shows minimal change in pH compared to formulation without cysteine.

### Effect of Cysteine on Globule Size Reduction

TABLE 29
Clevidipine Composition for Testing Effect of Cysteine

| | 40023-114, | |
|---|---|---|
| | 40023-115 | |
| | 40023-116 | |
| Ingredient | Qty/mL | |
| Clevidipine | 0.5 | mg |
| Egg Phosphatidylcholine | 12 | mg |
| Soybean oil | 200 | mg |
| Glycerine | 22.5 | mg |
| Oleic acid | 0.3 | mg |
| Methyl paraben | 0.08 | mg |
| Alpha Tocopherol | 0.1 | mg |
| L-cysteine HCL | 0.1 | mg |
| Milli-Q-Water | 764.1 | mg |

Process: Same as 40023-042, but dispersion of egg phosphatidylcholine in glycerin was mixed with water with methyl paraben before oil and water phases were mixed to form coarse emulsion. Cysteine was added after milling through microfluidizer for 5 cycles.

Results: Given in Table-30

TABLE 30
Effect of Cysteine on Globule Size During Milling Process

| | Mean Globule Size | | Mean Globule Size after 1 cycle | |
|---|---|---|---|---|
| | after 5 cycles at | | at 25 PSI after addition of | |
| Formulation | 25 PSI | | Cysteine | |
| 40023-114 | 208.3 | nm | 136.6 | nm |
| 40023-115 | 197.5 | nm | 126.9 | nm |

| | | | |
|---|---|---|---|
| 40023-116 | 139 | nm | 115 |

### Conclusion:

After 5 cycles at 25 PSI, mean size globule was about 200 nm. The size was almost same even after 2 cycles.
However, after addition of cysteine, mean globule size reduced by about 70 nm just after 1 cycle at 25 PSI.

### Example-11: Clevidipine Emulsion with 1 mg/mL of Clevidipine

| Ingredient | Qty/mL | |
|---|---|---|
| Clevidipine | 0.5 | mg |
| Egg Phosphatidylcholine | 12 | mg |
| Soybean oil | 200 | mg |
| Glycerin | 22.5 | mg |
| Oleic acid | 0.3 | mg |
| Methyl paraben | 0.08 | mg |
| Alpha Tocopherol | 0.2 | mg |
| L-cysteine HCL | 0.1 | mg |
| Milli-Q-Water | 764.1 | mg |

Advantages: The emulsion with 1 mg/mL Clevidipine will offer benefits of lower volume going to blood and lower lipids going into blood. Volume restrictions due to safety reasons might not restrict administration of higher dose of Clevidipine here.

### Overall Observations:

Overall, the development of Clevidipine Injectable emulsion showed many interesting and surprising results.

L-Cysteine was found to help improve chemical stability of the Clevidipine and physical stability of emulsion as well.

Emulsions with L-Cysteine were found to maintain physical stability of emulsion and those without L-Cysteine show phase separation in short time during stability testing at the higher temperature (40° C./75% RH).

L-Cysteine helped terminally sterilize emulsion with commonly used autoclave without the need of rotary autoclave. To maintain physical stability of the emulsion, and no phase separation, autoclave with option of mixing/rotation are essential. However, inclusion of the L-Cysteine, prevented such need.

For better stability, L-Cysteine must be added to the final emulsion after passing through microfluidizer or just one or two cycles before completion of milling.

L-Cysteine if used at concentrations >0.5 mg/mL adversely affects the stability of the Clevidipine in the emulsion, and higher amount of the impurities are observed during the stability when included in amounts exceeding 0.5 mg/mL.

Tocopherol has synergistic effect on stability if used along with L-Cysteine.

Surprisingly, Tocopherol found to reduce degradation of Egg Phosphatidylcholine to Lyso-Phosphatidylcholine.

Both Methyl Paraben and Sodium Benzoate were found be suitable as preservative in emulsion to reduce growth of microorganisms in case of adventitious contamination.

However, sodium benzoate when used at concentration of 0.5 mg/mL leads to phase separation of the emulsion.

Physical stability of emulsion during the autoclaving was found to be dependent on the pH of the emulsion. The pH of the emulsion must be >7.5 if the autoclave cycle is at 121° C./20 minutes. Otherwise, there will increase in globule size after autoclaving. A rotary autoclave is warranted if pH<7.5 is needed.

The emulsion pH can be lower, between 6.5 to 7.2 if autoclaving is done at 115° C./20 minutes.

L-Cysteine helps to stabilize the emulsion at lower pH of 6.5-7.2 during autoclaving, which will complement Clevidipine stability, which is found to be stable at neutral and acidic pH.

L-Cysteine helps in stabilization of pH during the autoclaving.

L-Cysteine helps in globule size reduction during final milling cycle, which otherwise difficult and would more milling cycles.

Adventitious contamination testing revealed that formulations containing 0.08 mg/mL, or 0.05 mL methyl paraben or 0.1 mg/mL sodium benzoate are equally effective as Cleviprix® in reducing rate of growth of microorganisms in the experimental conditions tested.

What is claimed:

1. An injectable Clevidipine emulsion formulation, comprising:
a) Clevidipine;
b) an oil;
c) an phosphatidylcholine emulsifier;
d) a cosolvent, optionally comprising glycerol;
e) water;
f) a coemulsifier;
g) optionally, an antioxidant, and
h) a preservative
wherein the emulsion formulation exhibits enhanced stability at ambient conditions.

2. A Clevidipine emulsion formulation of claim 1, comprising:
a) from about 0.01 to about 0.1% W/W Clevidipine Butyrate;
b) from about 15 to about 25% W/W soya bean oil;
c) from about 1 to about 1.6% W/W of egg or soya phosphatidylcholine emulsifier;
d) from about 1 to about 5% W/W of Glycerol;
e) from about 65 to about 80% W/W of water;
f) from about 0.01 to about 0.04% W/W sodium oleate or Oleic acid;
g) from about 0.01 to about 0.05% W/W Tocopherol
h) from about 0.001 to 0.05% W/W Cysteine and
i) from about 0.001 to 0.05% W/W preservative.

3. The injectable Clevidipine emulsion formulation of claim 1, wherein the oil is soya bean oil.

4. The injectable Clevidipine emulsion formulation of claim 1, wherein the amount of oil in the formulation is from about 15 to about 25% W/W of the formulation.

5. The injectable Clevidipine emulsion formulation of claim 1, wherein the phosphatidylcholine emulsifier is either from an egg or a soya source.

6. The injectable Clevidipine emulsion formulation of claim 1, wherein the amount of the phosphatidylcholine emulsifier in the formulation is from about 1 to about 1.6% W/W of the formulation.

7. The injectable Clevidipine emulsion formulation of claim 2, wherein the emulsion is an oil in water emulsion, formed by combining a water phase with a clevidipine-containing oil phase to form a first emulsion and wherein the cysteine is added to either the final emulsion or one or more milling cycles before completion of milling to form the injectable Clevidipine emulsion formulation.

8. The injectable Clevidipine emulsion formulation of claim 1, wherein the antioxidant is L-cysteine or tocopherol or mixture thereof.

9. The injectable Clevidipine emulsion formulation of claim 1, wherein the amount of antioxidant in the formulation is from about 0.01 to about 0.05% W/W of the formulation.

10. The injectable Clevidipine emulsion formulation of claim 1, wherein the cosolvent is a short chain alcohol, optionally selected from the group consisting of glycerol, ethanol, benzyl alcohol, propylene glycol, polymeric alcohol, and polyethylene glycol.

11. The injectable Clevidipine emulsion formulation of claim 10, wherein the cosolvent is glycerol.

12. The injectable Clevidipine emulsion formulation of claim 1, wherein the amount of the cosolvent in the formulation is from about 1 to about 5% W/W of the formulation.

13. The injectable Clevidipine emulsion formulation of claim 1, wherein the amount of the water in the formulation is from about 65 to about 80% W/W of the formulation.

14. The injectable Clevidipine emulsion formulation of claim 1, wherein the co-emulsifier is a salt of a fatty acid.

15. The injectable Clevidipine emulsion formulation of claim 14, wherein the co-emulsifier is oleic acid or sodium oleate.

16. The injectable Clevidipine emulsion formulation of claim 1, wherein the amount of the co-emulsifier is from about 0.01 to about 0.04% W/W of the formulation.

17. The injectable Clevidipine emulsion formulation of claim 1, wherein the initial pH of the composition is greater than or equal to at least 7.0.

18. The injectable Clevidipine emulsion formulation of claim 1, wherein the initial pH of the composition is at least about 7.5.

19. The injectable Clevidipine emulsion formulation of claim 1, wherein the preservative is a methyl paraben or sodium benzoate or a mixture thereof.

20. The injectable Clevidipine emulsion formulation of claim 1, wherein the amount of preservative is 0.01 to 0.05% W/W.

21. The injectable Clevidipine emulsion formulation of claim 1, wherein stable at 40° C./75% RH for at least 1 month and at least 6 months at 25° C./60% RH.

22. A method of preparing a clevidipine emulsion having extended stability, comprising:
a) providing an aqueous phase comprising water and optionally one or more of tonicity agents, pH adjusting agents, buffers, emulsifiers, cosolvents, buffer;
b) providing an oil phase which includes an oil, clevidipine, oleic acid and optionally a first antioxidant, a buffer, a pH adjusting agent;
c) combining the oil phase with the aqueous phase under conditions sufficient to form a first emulsion;
d) combining the coarse first emulsion with a stabilizing amount of L-cysteine; and
e) subjecting the resultant mixture to high shear mixing; and recovering a final clevidipine emulsion having extended stability.

23. The method of claim 22, wherein stabilizing amount of L-cysteine is from about 0.1 to about 0.5 mg/ml based upon the volume of the first coarse emulsion.

24. The method of claim 23 wherein the high shear mixing is carried out by microfluidization or milling.

25. The method of claim 22, wherein the pH of the emulsion is from about 6.5 to about 8.5.

26. The emulsion of claim 1, wherein the globule size of final emulsion is in the range of 100-180 nm.

27. The clevidipine emulsion composition prepared by the method of claim 22.
